(19) United States
(12) Plant Patent Application Publication
(10) Pub. No.: US 2004/0168235 P1
(43) Pub. Date: Aug. 26, 2004

(54) Apple tree named 'Rosy Glow'

(76) Inventors: Harleigh C. Mason, South Australia (AU); Ashley G. Mason, South Australia (AU)

Correspondence Address: KLARQUIST SPARKMAN, LLP, One World Trade Center, Suite 1600, 121 S.W. Salmon Street, Portland, OR, 97204, US

(21) Appl. No.: 10/712,783

(22) Filed: Nov. 12, 2003

Related U.S. Application Data
(63) Continuation of application No. 10/160,991, filed on May 31, 2002, now abandoned.

(51) Int. Cl. A01H 5/00

(52) U.S. Cl. PLT/161

(57) ABSTRACT

A new and distinctive variety of an apple tree, named ‘Rosy Glow,’ characterized by highly colored fruit, including the fruit which is produced in shaded parts of the tree and in districts that are not conducive to good red color, early red coloring of the fruit allows it to be picked at optimum maturity for long-term storage, and fruit having a red color extending into the calyx.

### CROSS REFERENCE TO RELATED APPLICATIONS

None.

### STATEMENT REGARDING FEDERALLY SPONSORED RESEARCH OR DEVELOPMENT

No U.S. federally sponsored research or development.

### LATIN NAME OF THE GENUS AND SPECIES OF THE PLANT CLAIMED

Malus domestica

### VARIETY DENOMINATION

‘Rosy Glow’

### BACKGROUND OF THE INVENTION

The present invention relates to a new and distinct variety of apple tree named ‘Rosy Glow,’ which was discovered by the inventors in a cultivated area of an existing orchard of ‘Cripps Pink’ (U.S. Pat. No. 7,880) trees being grown at Masons Road, Forest Range, South Australia, Australia. The ‘Cripps Pink’ trees had been planted in September 1992. On Apr. 22, 1996, the inventors noticed one limb having red-colored apples on one of the ‘Cripps Pink’ trees. At the same time, the apples on the remaining branches of this tree were poorly colored (mostly green with very little red).

The ‘Rosy Glow’ variety differs from other apple trees known to the inventors, including ‘Cripps Pink’. The ‘Rosy Glow’ variety produces highly colored fruit in shaded parts of the tree and in districts that are not conductive to good color. In addition, the fruit of the ‘Rosy Glow’ variety colors earlier than ‘Cripps Pink’, allowing the fruit of the new ‘Rosy Glow’ variety to be picked at optimum maturity for long-term storage. In addition, the fruit of ‘Rosy Glow’ trees has red color extending even into the calyx.

### BRIEF SUMMARY OF THE INVENTION

The first asexual propagation of this new variety was performed in April 1996. Budwood was selected from the original limb mutation and grafted by one of the inventors onto ‘Northern Spy’ (unpatented) rootstock in an orchard on Masons Road, Forest Range, South Australia. These asexually propagated trees, the original limb mutation, and other ‘Rosy Glow’ trees since asexually propagated continue to produce highly colored fruit and all other desirable characteristics of ‘Rosy Glow.’ This demonstrates that the foregoing and all other characteristics and distinctions come true to form and are established and transmitted through succeeding asexual propagations. Moreover, the new ‘Rosy Glow’ apple variety exhibits good union between the root-stock and the grafting stock with no rejection tendencies observed to date.

Certain characteristics of this variety, may change with changing environmental conditions (e.g., photoperiod, temperature, moisture, soil conditions, nutrient availability, or other factors). For example, leaf colors may be brighter green if the trees are grown in soil with greater nitrogen concentrations, and may be more yellow when grown in soil containing lesser amounts of nitrogen. Color descriptions and other terminology are used in accordance with their ordinary dictionary descriptions, unless the context clearly indicates otherwise. Color designations (hue/value/chroma) are made with reference to The Royal Horticultural Society Colour Chart (RHS).

### BRIEF DESCRIPTION OF THE PHOTOGRAPHS

The accompanying color photographs show typical specimens of the new tree, leaves, and fruit of this new apple tree variety and depict the color as nearly true as is reasonably possible to make the same in a color illustration of this character. It should be noted that colors may vary, for example due to lighting conditions at the time the photographs are taken. Therefore, color characteristics of this new variety should be determined with reference to the observations described herein, rather than from the photographs alone.

FIG. 1 shows the ‘Rosy Glow’ mutation branch (red apples) on a ‘Cripps Pink’ tree together with substantially green apples from another branch of this tree).

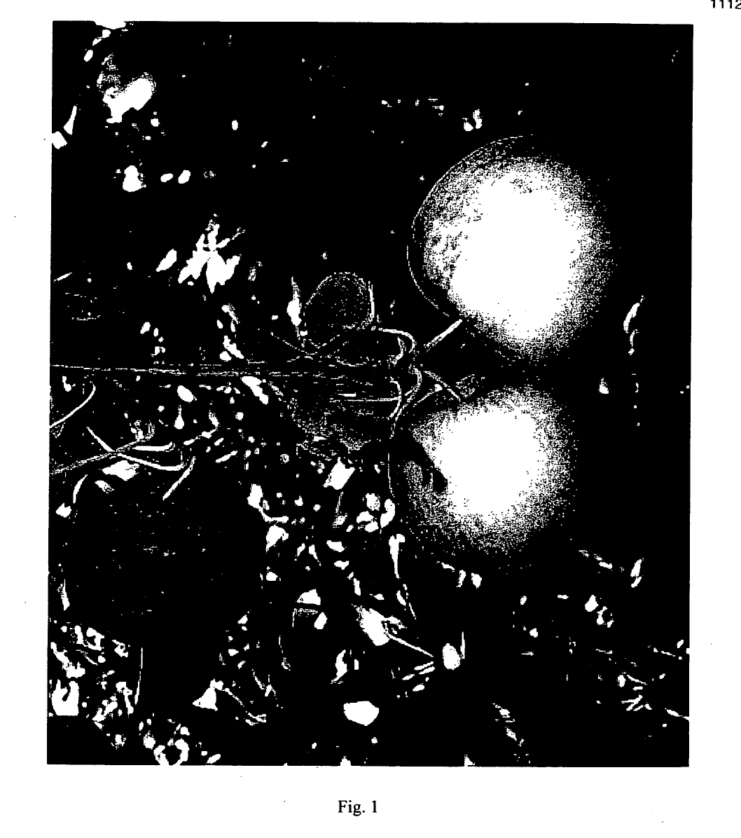

FIG. 2 shows a tree of the ‘Rosy Glow’ variety demonstrating excellent, even color of the fruit over the entire tree.

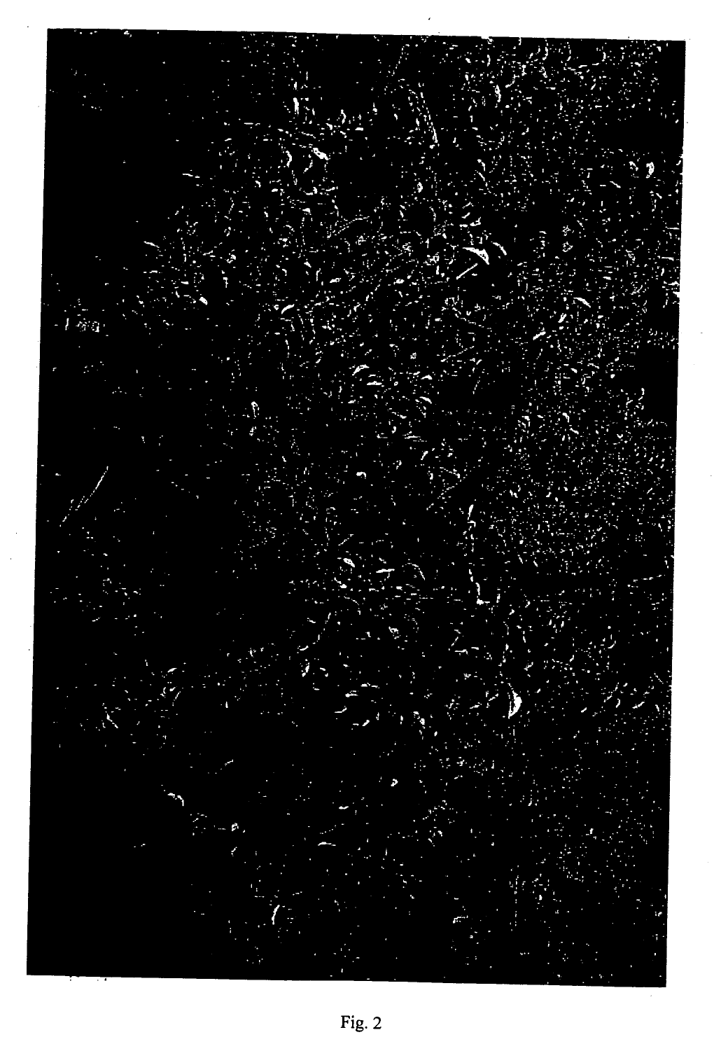

FIG. 3 shows a close-up view of the ‘Rosy Glow’ apples on the ‘Rosy Glow’ tree.

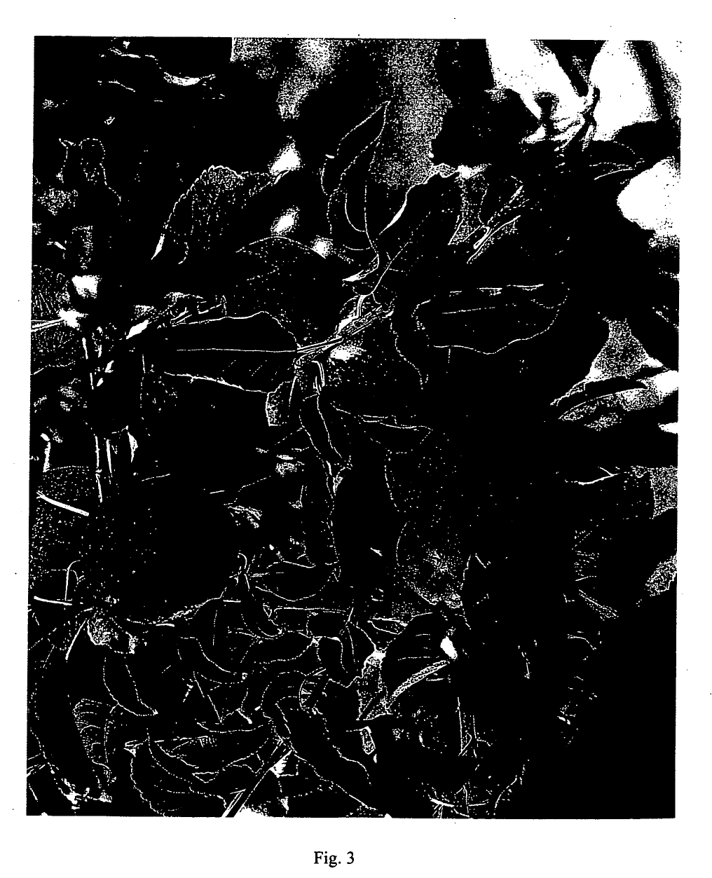

FIG. 4 shows ‘Rosy Glow’ apples, including cross-sections thereof.

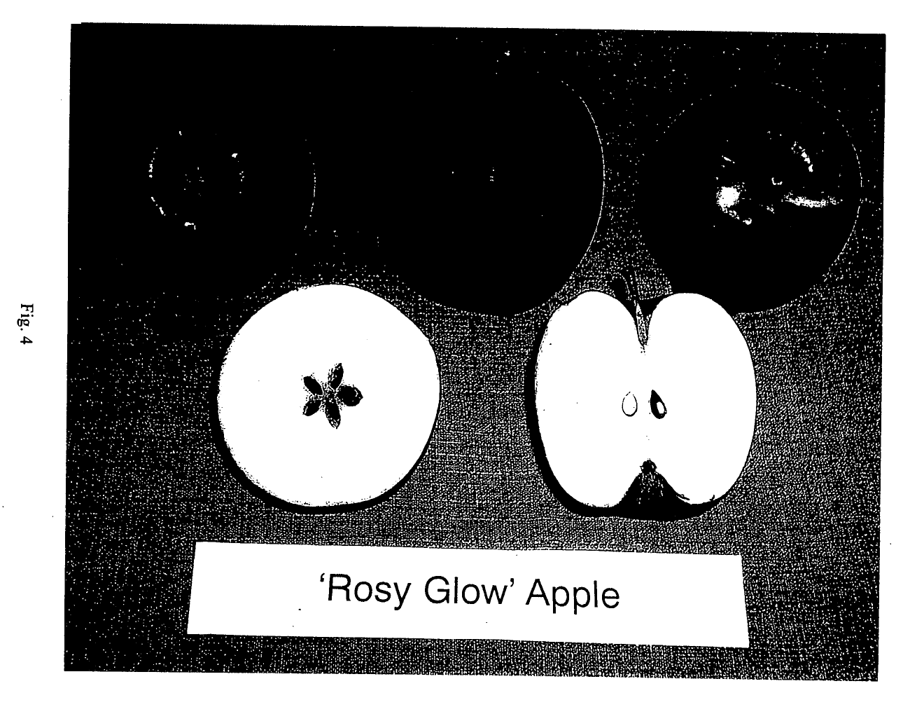

### DETAILED BOTANICAL DESCRIPTION

The following detailed description of the ‘Rosy Glow’ cultivar apple tree is based on observations of asexually propagated progeny thereof, growing in Taggerty, Victoria, Australia. Specifically, the observations described below are of trees in their fourth summer from grafting onto ‘Northern Spy’ rootstock.

Scientific name: Malus domestica ‘Rosy Glow’.

Parentage: Limb sport of ‘Cripps Pink’ Malus domestica.

Botanical description:

Tree:

Vigor.—Medium.

Overall shape.—Upright to slightly fastigate.

Height.—Height of the observed trees ranged from about 9 to about 10 feet.

Width.—Overall spread of the observed trees was about 6 feet.

Trunk: Medium stocky.

Trunk bark texture.—Medium rough with some ridging.

Trunk bark color.—At four years of age bark color was greenish-grey-cream, like RHS 195B.

Size.—Caliper about 8 inches at 24 inches above the ground.

Trunk markings.—Numerous lenticels about one-fourth to one-half inch longand some pimple-like protrusions. The lenticels are the same color as the trunk, and are slightly elongated in shape.

Branches:

Primary branches.—Upright and spreading. Branch angle at emergence typically is about 50 to about 65 degrees from the vertical. The angle does not vary from the top to the bottom of the tree. Some included back in branch notches. During one year, limbs from trees growing in Taggerty, Victoria, Australia exhibited growth ranging from 15 inches to 30 inches and the average growth was 24 inches.

Branch color.—Color of one year branch is green, like RHS 144B; two-year-old branch color is green-brown, like RHS 199B.

Branch pubescence.—Observed on new and older branches as grey-white, like RHS 196D.

Branch lenticels.—Shape: Round, becoming elongated with age. Quantity: Medium density. About 38 per square inch. Color: light brown, like RHS 164D. Size: Medium, 1/16 inch in diameter.

Internodes: Average internode length on one-year-shoot about 1 1/16 inches.

Bearing: Annual.

Cold hardiness: Unknown. Trees growing in Taggerty, Victoria, Australia, which rarely gets below 27° C.

Leaves (from observations of 20 typical leaves in November of 2001.):

Form.—shape Generally oval shape.

Texture.—Medium thick.

Sheen.—Medium dull.

Length.—About 3.5 in to about 5.0 in, averaging about 4.25 in.

Width.—About 2.25 in to about 2.75 in, averaging about 2.5 in. Petiole: Length is about 1 2/16 inches and about 1/16 inch in diameter. Color of petiole is red-green (red like RHS 179A, and green like RHS 148D).

Margin.—Irregularly serrated.

Tip.—Acutely pointed.

Leaf color.—Upper surface is green, like RHS 137A. Underside of leaf is light green, like RHS 147B. Vein color is light green, like RHS 147C.

Pubescence.—Upper surface: pubescence present towards the tip, white-translucent in color. Lower surface: pubescence present on the entire surface and the veins. White translucent to very slightly grey brown in color, like RHS 160D.

Stipules.—Prominent, curved. In a set of observed samples, mean length ¾ in., mean width ⅛ in.

Bloom time: In 2001, first bloom in Taggerty, Victoria, Australia was Sep. 28, 2001, full bloom was Oct. 4, 2001, and petal drop was Oct. 10, 2001.

Flowers (observations are from a sample of typical flowers.):

Size.—Individual flowers are medium size of about 1.25 inches.

Shape.—Rounded.

Time of bloom.—Early to mid season. Depends on the amount of chill units received the previous winter.

Bloom description.—Unopened Bud: pink, line RHS 67B. Fully open flower: White to very pale pink, like RHS 155D, with pale pink RHS 65D. In general, the flowers have a white ground color with a pink blush which fades as the blossom ages.

Petals.—Rounded to ovate, about ⅝ inch in length. Color is white, like RHS 155D, with 5 petals per flower.

Stamen.—Arranged in two rows. About 16 stamens, each about ⅜ inch in length. Color is cream white, like RHS 155D.

Anthers.—Pale yellow, like RHS 6D.

Pistil.—Stigma: Curved and flattened at the top, flattened at the base. Approximately 5/16 inch long. Style: Five fused at base, pale brown in color, like RHS 162D.

Sepals.—Recurved, light green (RHS 142D), with pubescence. Sepals are about ¼ inch in length, about ⅛ inch wide at base.

Fragrance.—Light.

Pollen.—Yellow, like RHS 6D.

Pollination requirements.—Same as ‘Cripps Pink’ or any diploid that blossoms in the same season.

Fruit (unless otherwise noted, from observations of 20 applies in November 2001.):

Shape.—Round oblong, usually asymmetric; a medium amount of ribbing with non-prominent lobes at calyx end.

Size.—Average box size, about 2.75 inches to about 3⅜ inches.

Length.—diameter ratio for fruit: the length to diameter ratio ranged from 1:1 to 1:11 and averaged 1:1.04; mean height 2.76 inches mean diameter 2.86 inches (for 20 typical apples grown in Taggerty, Victoria, Australia harvested on May 2, 2002).

Stem cavity.—Medium broad to deep, about ⅞ inch wide by about 11/16 inch deep.

Basin cavity.—Medium depth and width. About ⅞ inch wide and about 7/16 inch deep. Pubescence present.

Stem.—Predominantly short-stout with some variability, about ⅝ inch in length by about ⅛ inch thick; reddish brown to greenish brown (RHS 175B) from observations on May 2, 2002.

Core.—Bundle Area: large, unsymmetrical at base, unequal halves of area. Bundles: greenish-white, slightly conspicuous. Core lines: meeting.

Seeds.—8 perfect seeds, 0 imperfect.

Locules (carpels).—Mostly closed with some partly open. Five in number.

Skin.—Medium thick; glossy finish; medium tendency to become oily or waxy in storage if over mature when picked. No evident cracking tendency observed with apple maturity.

Russet.—Absent.

Lenticels.—Round-shaped lenticles. Color is whitish-cream like RHS 158C. Range in size from 1/64 inch to 1/16 inch (mean 1/32 inch).

General color effect.—Attractive solid red-pinkish red blush over yellow ground color. Very distinctive.

Ground color.—Green to yellow, like RHS 10B at last pick. More yellow with last pick as fruit becomes over-mature.

Overcolor.—General color is bright red-pink solid blush with very slight darker red-pink striping. Color like RHS 47A to 50A. Controlled Atmosphere Storage fruit characteristics at 7 months from harvest. Acid content: Medium. Firmness: 19-21 pounds; mean firmness 20 pounds (Observations of 20 fruit harvested May 2, 2002). Soluble solids: 18% (Observations of 20 fruit harvested May 2, 2002). Flavor: sweet to tart. Juiciness: medium. Flavor: mild. Aroma: mild, pleasant. Flesh color: creamy white, like RHS 4D.

Seed.—Acute in shape, typically two per cell, occasionally one; brown like RHS 174A, about ⅜ inch in length by about 3/16 inch wide.

Fruit production.—First picking date in Taggerty, Victoria, Australia, about April 7, and last picking date was about April 14. In a sampling of 10 trees growing at Taggerty, Victoria, Australia, and during the 2001 harvest, an average of about 80 lbs of fruit were obtained per tree. The trees were 4 years old and budded on ‘Northern Spy’ rootstock.

Keeping quality.—Good, quality maintained in controlled atmosphere storage for about eight months. About four months in common storage at 0° C.

Usage.—Good for fresh eating and dessert purposes.

Disease and insect resistance/susceptibility: Susceptible to black spot. Like the susceptibility and tolerance as ‘Cripps Pink’.

We claim:

1. A new and distinct variety of apple tree named ‘Rosy Glow’ substantially as illustrated and described.
